(19) United States
(12) Patent Application Publication
(10) Pub. No.: US 2007/0078295 A1
(43) Pub. Date: Apr. 5, 2007

(54) Surgical implant system for treating female urinary incontinence

(76) Inventor: Susanne Landgrebe, Sullfeld (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/550,930

(22) PCT Filed: Apr. 5, 2004
(86) PCT No.: PCT/EP04/03598

§ 371 Date: Nov. 2, 2006

Related U.S. Application Data
(60) Provisional application No. 60/460,492, filed on Apr. 3, 2003.

(51) Int. Cl. A61F 13/00; A61F 2/00

(52) U.S. Cl. 600/37

(57) ABSTRACT

A surgical implant system for treating female urinary incontinence has at least one suspending device (10, 10″) and a support structure (100) for the urethra (182). The suspending device has an upper end area and a lower end area the upper end area being able to be fastened an on internal anatomical tissue structure (180) to the side of and above the urethra. The support structure is designed for implantation transversely with respect to the urethra and has a fastening device (104, 105) which is assigned to the suspending device and which fastens the support structure an the suspending device. The fastening device is arranged to the side of the urethra and, after it has been removably fastened on the suspending device, it can be moved along the suspending device in order to adjust the position of the support structure.

The invention relates to a surgical implant system for treating female urinary incontinence.

One procedure for treating female urinary incontinence is referred to as the TVT surgical technique and is described, for example, in WO 96/06567 and WO 97/13465. In this method, a surgical instrument is used in which in each case a strongly curved surgical needle is fastened to the two ends of a polypropylene implant tape and is guided with the aid of a removable handle. The two needles are guided through the vagina on opposite sides of a patient's urethra along the back of the pubic bone to the outside of the abdominal wall. The tape comes to lie in an arc under the urethra. The two ends of the tape are pulled through the abdominal wall and cut. They do not generally need to be sewn since the tape, because of its structure, acquires a good hold in the tissue and grows relatively quickly into the tissue. In the stress-free state, the tape does not touch the urethra, but, in the event of a stress, it provides a supporting action. Urinary incontinence can be treated quickly and effectively by this procedure.

Although the operation does not generally place much burden on the patient, it does however require skill on the part of the operating surgeon so as to avoid undesired injuries when passing the needles through. The healing process takes some time, and visible scars remain on the abdominal wall. In addition, the tape has to be relatively long, with the result that a not inconsiderable amount of foreign material is implanted in the body.

WO 02/30293 discloses a procedure for treating female urinary incontinence in which a surgical implant with a support structure for the urethra is introduced through an incision in the upper vaginal wall and is suspended on two tapes which are fastened, on both sides of and above the urethra, with the aid of tissue anchors on internal anatomical tissue structures behind the pubic bone. It is therefore not necessary to pass through the abdominal wall. Tissue structures which can be used for receiving the tissue anchors are in particular the rectus sheath behind the pubic bone or connective tissue in the retropubic space. This surgical technique has the same effect on the urethra as the aforementioned TVT procedure, but it avoids wounds on the abdominal wall and generally uses less implant material.

For the operation to be successful, it is important that the support structure under the urethra is correctly positioned. In the TVT procedure, this can be done simply by pulling-on the ends of the tape which protrude from the abdominal wall. However, in the procedure known from WO 02/30293, the exact positioning of the support structure is difficult and time consuming. In one design, the support structure under the urethra has an opening through which the lower areas of the tapes of the suspending device are pulled from both sides and are fastened there by fitting a ring. This has to be done in a very confined space and, after the fastening has been done, the position of the support structure cannot easily be corrected. In another design, the lateral edge areas of the support structure in each case have a number of slits or round openings through which the lower tape areas of the suspending devices are pulled through repeatedly and thereby diverted. This too is difficult and prevents simple correction of the position of the support structure after it has been fastened.

The object of the invention is to make it possible, in the treatment of female urinary incontinence, for a urethral support structure to be able to be fastened on internal anatomical tissue structures so that wounds on the abdominal wall are avoided, and in so doing to simplify the surgical procedure so that the correct position of the support structure can be obtained without problems.

This object is achieved by a surgical implant system for treating female urinary incontinence having the features of claim 1. Advantageous embodiments of the invention are set out in the dependent claims.

The surgical implant system according to the invention for treating female urinary incontinence has at least one suspending device with an upper end area and a lower end area, the upper end area being able to be fastened on an internal anatomical tissue structure to the side of and above the urethra. Terms such as “above”, “below” and “to the side of” refer here, and in the rest of the text, to the implanted state in a patient's body. The system additionally has a support structure for the urethra, which is designed for implantation transversely wit respect to the urethra. The support structure has a fastening device which is assigned to the suspending device and which fastens the support structure on the suspending device. The fastening device is avenged (in the implanted state) to the side of the urethra. After it has been fastened on the suspending device, the fastening device can be moved along the suspending device in order to adjust the position of the support structure.

Anatomical tissue structures on which the suspending device can be fastened via its upper end area are, for example, the pubocervical fascia, the tissues mentioned in WO 02/30293, and other strong tissue structures. After the upper end area of the suspending device is fastened on the tissue structure, the remaining area of the suspending device is situated to the side of the urethra. There, it can be easily accessed by the operating surgeon, so that the fastening device which is assigned to the suspending device, and which is likewise situated to the side of the urethra, can be fastened on the suspending device without any problem. In contrast to the already known implant, however, this fastening of the support structure on the suspending device is not rigid, and instead the fastening device can be moved along the suspending device, so that the position of the support structure can be easily and safely adjusted within a readily accessible operating field. This makes the surgical procedure considerably easier. The correct position of the support structure is reached when, sunder application of stress, urine no longer escapes. A stress situation can be simulated during the operation by asking the patient to cough.

The suspending device preferably has a threadlike structure with an upper end and a lower end. In a preferred embodiment, the threadlike structure is a monofilament, for example of polypropylene. Depressions can in this case be arranged along the monofilament, these depressions being designed for engagement of a locking projection provided on the fastening device. The distances between the depressions are preferably chosen such that the position of the support structure is slightly less propitious than the optimum position when the locking projection of the fastening device is not lying in the depression provided for the optimum position but is lying in a depression immediately adjacent thereto. The form-fit connection between locking projection and depression provides for a secure arrest of the fastening device on the suspending device, However, under the effect of a sufficient force (which should be greater than the forces acting on the support structure during stress), the locking projection can leave the depression so that the fastening device can be moved along the suspending device. In the course of the healing process, the support structure grows into the body tissue, so that the forces exerted on the fastening device and on the suspending device diminish anyway.

The locking projection of the fastening device can be arranged on a spring tongue. In an advantageous embodiment, two locking projections lying opposite one another can be moved away from one another by lateral pressure exerted on the fastening device. In this case, the fastening is particularly secure because such a lateral pressure on the fastening device does not occur under normal conditions, but only when so desired during the operation (for example using a suitable instrument).

The fastening device preferably has a ring-shaped or sleeve-shaped element which can be provided with one or more locking projections This element can preferably be pushed over the lower end area of the suspending device onto said suspending device.

In another preferred embodiment of the system according to the invention, the fastening device has a loop of threadlike material. In this case, the suspending device preferably has a threadlike structure with an upper end and a lower end (for example as a monofilament), and the loop is designed as a double loop having a first loop and a second loop. The first loop in the edge area of the support structure is pulled through the support structure. The second loop is provided to receive the threadlike structure of the suspending device, this threadlike structure for example being able to be passed twice trough the second loop. The double loop as a whole can be pulled tight via the end portions of the threadlike material which issue from the first loop. In this way, the threadlike structure is connected tightly and securely to the support structure. Because of the design of the double loop, it is however still possible to move the support structure along the suspending device in order to obtain the optimum position.

The support structure preferably has a tape which is designed for implantation transversely with respect to the urethra Such a tape as support structure is in principle known from the TVT procedure discussed at the outset In the system according to the invention, however, the tape is shorter since it does not have to reach as far as the abdominal wall of the patient but instead only over the area of the urethra and the adjacent zone and, if appropriate, at one end, as far as the anatomical tissue stuce provided for fastening it (see below). The fastening device discussed above is preferably arranged in the area of an end of the tape. The tape can for example be knit from a polypropylene monofilament, and, if the fasten device is likewise made of polypropylene, it can be easily connected to the tape by welding. Other designs are also conceivable.

In preferred embodiments, the suspending device has, in its upper end area, a tissue anchor. The tissue anchor is used to fasten the suspending device on the internal anatomical tissue structure. There are many possible designs of the tissue anchor. Thus, for example, it can have at least two wings which are preferably movable from a configuration extending substantially parallel to the longitudinal axis of the suspending device, to a configuration extending transversely with respect to the longitudinal axis of the suspending device. This movement is preferably automatic, so that the tissue anchor can be pushed from below through an opening in the anatomical tissue structure while the wings extend substantially parallel to the longitudinal axis of the suspending device, and the wings automatically position themselves transversely, and thereby anchor the tissue anchor in the tissue, as soon as they have passed completely through the opening.

The wings can for example be made of tape-like textile material. A ring-shaped, sleeve-shaped or wing-shaped structure of textile material arranged under the wings is advantageous, which structure, with the tissue anchor introduced, comes to lie within the opening in the tissue structure and there affords an additional hold. The textile material has pores or openings through which tissue grows in the course of the healing process, so that the tissue anchor is particularly securely fastened.

As has already been mentioned, the tissue anchor can have many possible structural forms. In further examples, it can be made up of a plurality of structural parts so that it can thus be optimally adapted to the tissue present in the individual case. This can be achieved, for example, if the suspending device has, at the upper end for example of the monofilament, a contact plate with a central extension onto which it is possible to attach anchor elements, for example wings made of textile material or a moulded part which can open out like an umbrella A cap placed on the upper end can be used to secure this.

In a preferred embodiment, the implant system according to the invention has two suspending devices, namely a first suspending device and a second suspending device, which are provided for fastening on the left side above the urethra and on the right side above the urethra, respectively. The support struggle in this case has two fastening devices, namely a first fastening device for the first suspending device and a second fastening device for the second suspending device. The two suspending devices and the two fastening devices are each preferably of identical structure. The system can in this way be configured symmetrically, which in principle facilitates the surgical procedure, because the surgical steps are performed in essentially the same way on the left and right of the urethra.

In an alternative embodiment, the system has only one suspending device of the type discussed, which is assigned a fastening device which can be moved along said suspending device. This suspending device is fastened on one side of the urethra, and the position of the support structure can be adjusted by moving the fastening device on the suspending device. A possibility of fastening the support structure must also be provided on the other side of the urethra, For this purpose, the support preferably has a tape which is designed for implantation transversely with respect to the urethra, said fastening device being arranged in the area of one end of the tape; the tape is continued in the other direction and carries a tissue anchor at its other end. In this case, therefore, on the other side, the tape itself assumes the function of a suspending device. This embodiment is particularly simple and cost-effective if the fastening device is designed as a double loop of the kind discussed above.

As has already been mentioned with reference to the tissue anchor, it is advantageous for components of the implant system according to the invention to be made available in different forms or designs, so that the components required in a particular case can be put together individually for am operation. Thus, for example, different sizes of the support structure can be provided. Colour markings, on the suspending device too, are useful for correct allocation.

As a further component, the system according to the invention can have a sliding instrument for moving a fastening device along a suspending device. The sliding instrument can for example have a tube with a proximal end and a distal end, the tube preferably being bent at a right angle and having, opposite an opening at the distal end, a further opening in the area of tie bend position. With the aid of such a sliding instrument, a fastening device placed on a suspending device can be moved in the longitudinal direction of the suspending device. In this case, the suspending device is guided through the inside of the tube from the opening at the distal end as far as the opening in the area of the bend position, and the fastening device, when advanced, bears on the distal end of the tube. In an alternative embodiment, the tube is not bent at a right angle but is instead provided with a preferably laterally offset handle, in which case the suspending device extends through the whole of the interior of the tube during use.

If the fastening device has been pushed too far up when setting the optimum position of the support structure, it is possible to pull the fastening device back down again. For this purpose, a grip instrument can be used with which the fastening device is gripped laterally, or a special instrument which for example has one or two angled claws which bear on the upper end of the fastening device so as to grip the latter from behind.

As a further component of the system according to the invention, a trocar instrument can be used for introducing a tissue anchor. Such a trocar instrument preferably has a sleeve with a proximal end and a distal end which can be bevelled but can also be straight A trocar is inserted into the sleeve via the proximal end, the tip of this trocar protruding at the distal end in the inserted state. In this state, the trocar instrument can be used to establish an opening at the position where a suspending device is intended to be anchored on an internal anatomical tissue structure. After the trocar has been pulled back in the proximal direction, the sleeve is located in the tissue and thus holds the opening open. The suspending device, preferably with the tissue anchor folded up or positioned parallel to the longitudinal axis of the suspending device, can then be advanced inside the sleeve until the tissue anchor emerges at the distal end of the sleeve and deploys or positions itself transversely. The sleeve can then be pulled back in the proximal direction.

The sleeve and the trocar are preferably bent, which makes handling easier in the given anatomical conditions. In an advantageous embodiment, the sleeve and the trocar each have a handle in the proximal end area, and the handles of the sleeve and of the trocar are preferably ergonomically matched to one another.

The invention is explained in more detail below with reference to illuative embodiments. In the drawings:

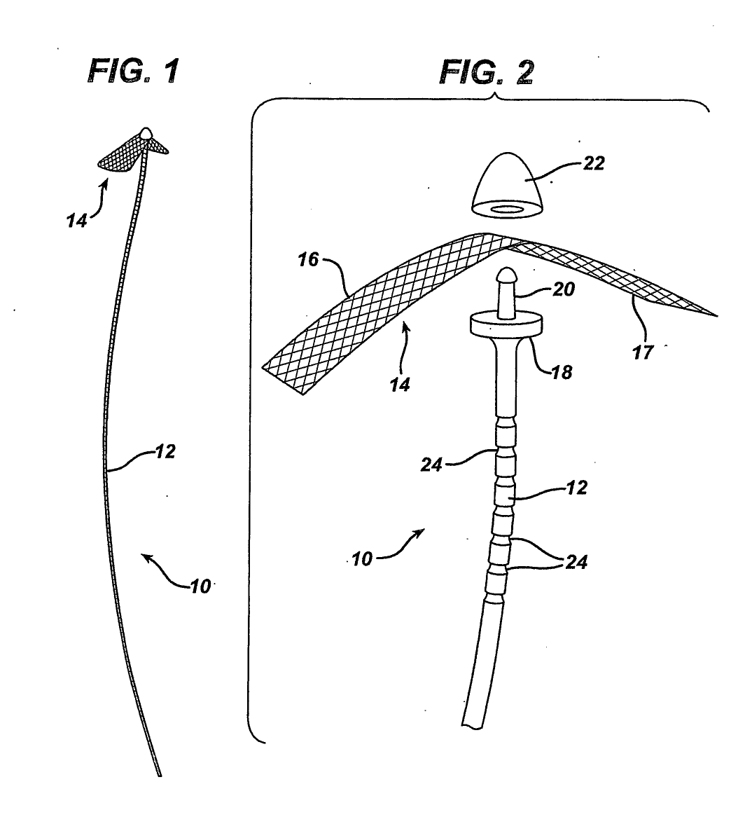

FIG. 1 shows a perspective view of an embodiment of a suspending device of the implant system,

FIG. 2 shows an enlarged exploded view of the upper area of the suspending device from FIG. 1,

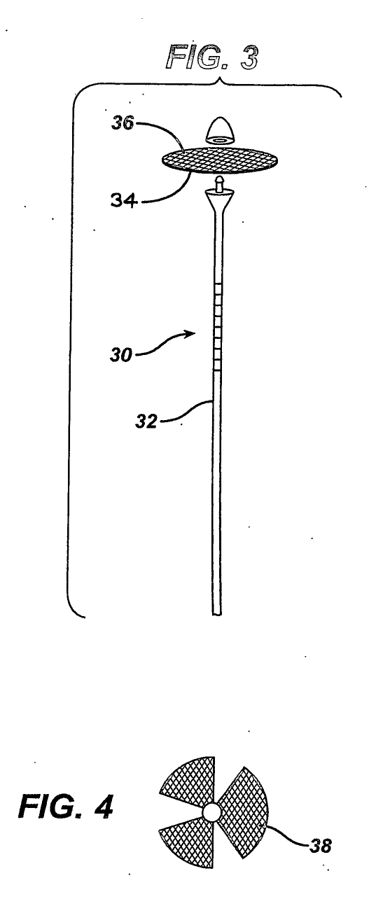

FIG. 3 shows an exploded view of a further embodiment of the suspending device,

FIG. 4 shows a plan view of the tissue anchor in a further embodiment of the suspending device,

FIG. 5 shows a perspective view of the upper area in a further embodiment of the suspending device, specifically, in part (a), with the tissue anchor folded up, and, in part (b), with the tissue anchor deployed,

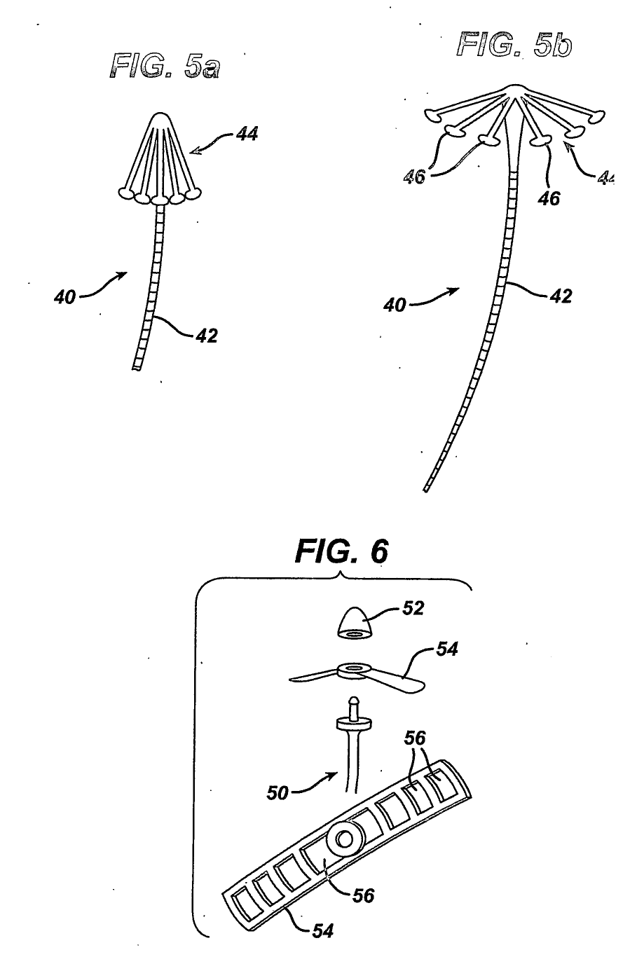

FIG. 6 shows a further enmbodiment of a suspending device, specifically, in part (a), a perspective exploded view of the upper area of the suspending device, and, in part (b), a plan view of the tissue anchor,

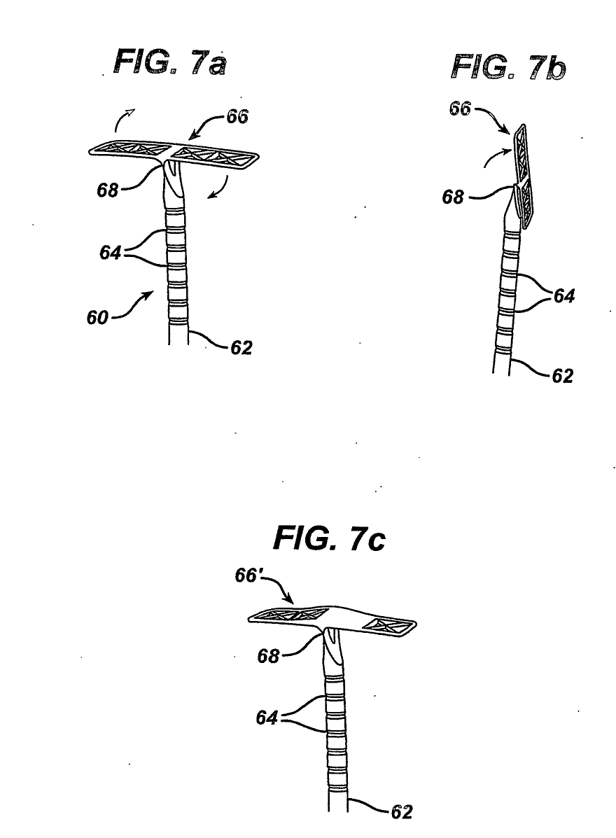

FIG. 7 shows a perspective view of a futher embodiment of the suspending device, specifically, in part (a), with the tissue anchor positioned transversely, and, in part (b), with the tissue anchor folded up, and, in part (c), with the tissue anchor positioned transversely in a slightly modified embodiment,

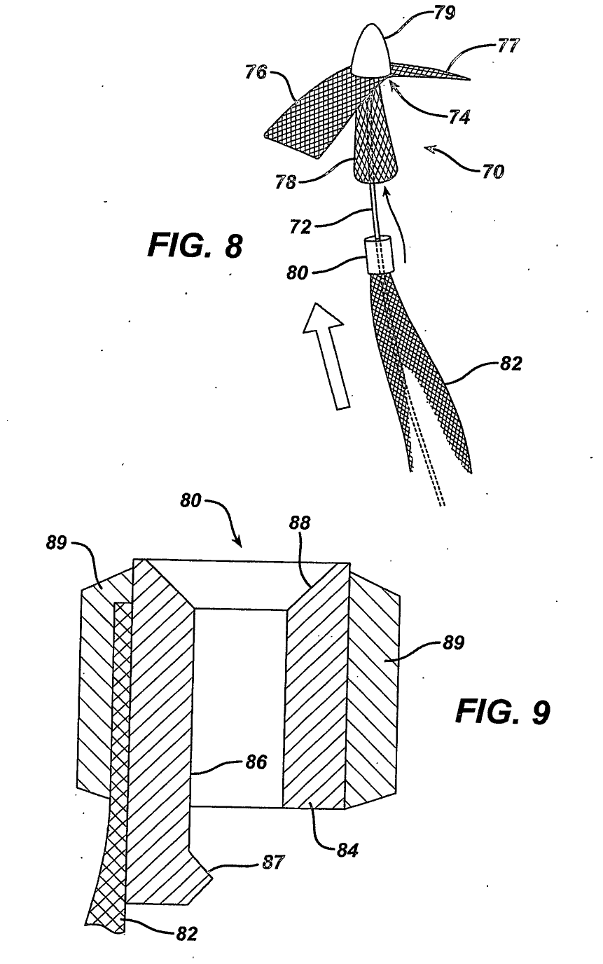

FIG. 8 shows a perspective view of a further embodiment of the suspending device, with a fastening device of a support structure of the implant system pushed onto the lower area of said suspending device,

FIG. 9 shows a longitudinal section through the fastening device from FIG. 8,

FIG. 10 shows a perspective view of the fastening device from FIGS. 8 and 9 on the suspending device from FIG. 8, the support structure having been omitted for reasons of clarity,

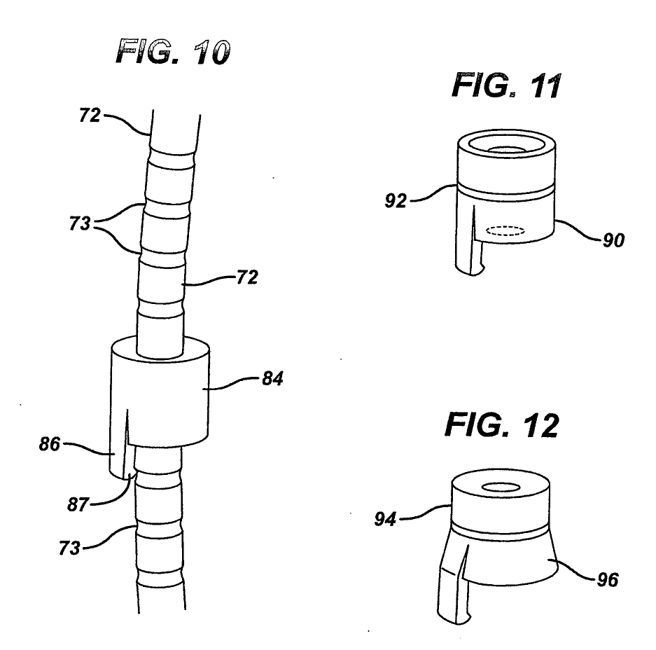

FIG. 11 shows a perspective view of a sleeve-shaped element in a further embodiment of the fastening device,

FIG. 12 shows a perspective view of a sleeve-shaped element in a further embodiment of the fastening device,

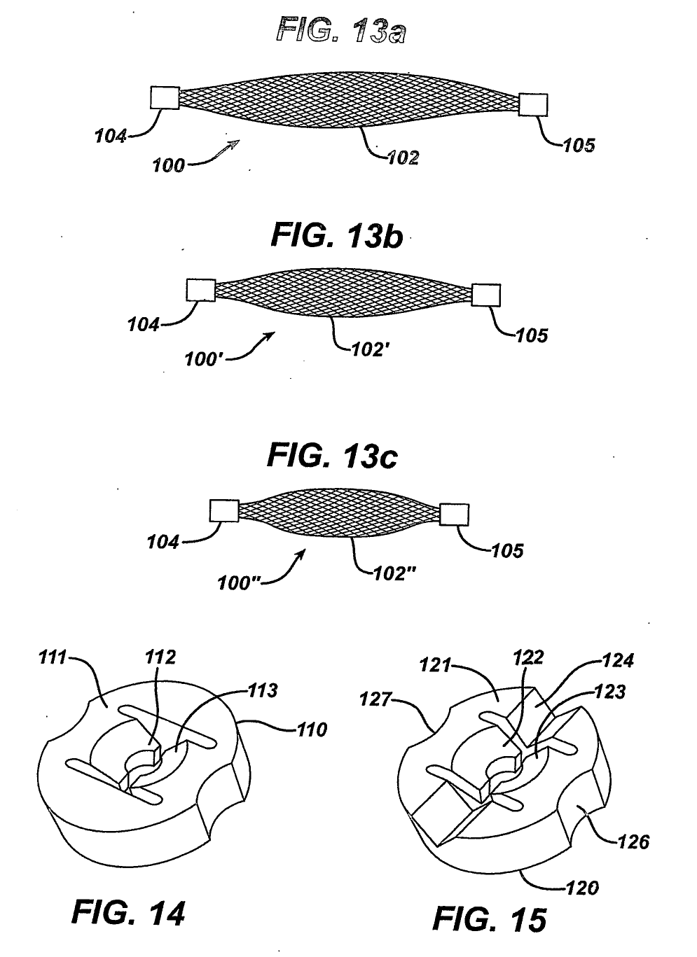

FIG. 13 shows, in parts (a), (b) and (c), plan views of support structures of the type shown in FIG. 8, and in different sizes,

FIG. 14 shows a perspective view of a further embodiment of the fastening device,

FIG. 15 shows a perspective view of a further embodiment of the fastening device,

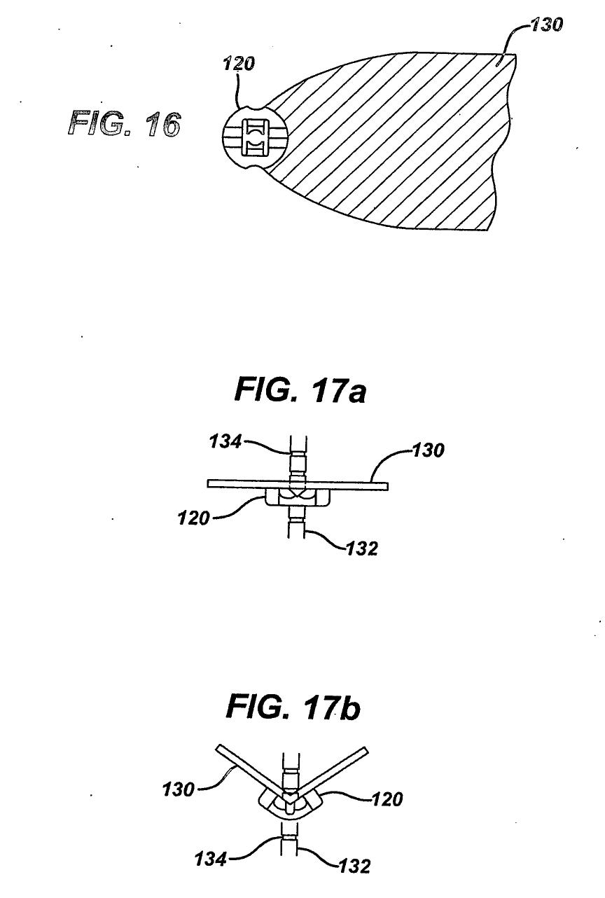

FIG. 16 shows a plan view of the end area of a support structure, with a fastening device according to FIG. 15 arranged there,

FIG. 17 shows, in part (a), a side view of the support structure from FIG. 16 locked on a monofilament of a suspending device, and, in part (b), an illustration of how the fastening device can be unlocked by lateral pressure,

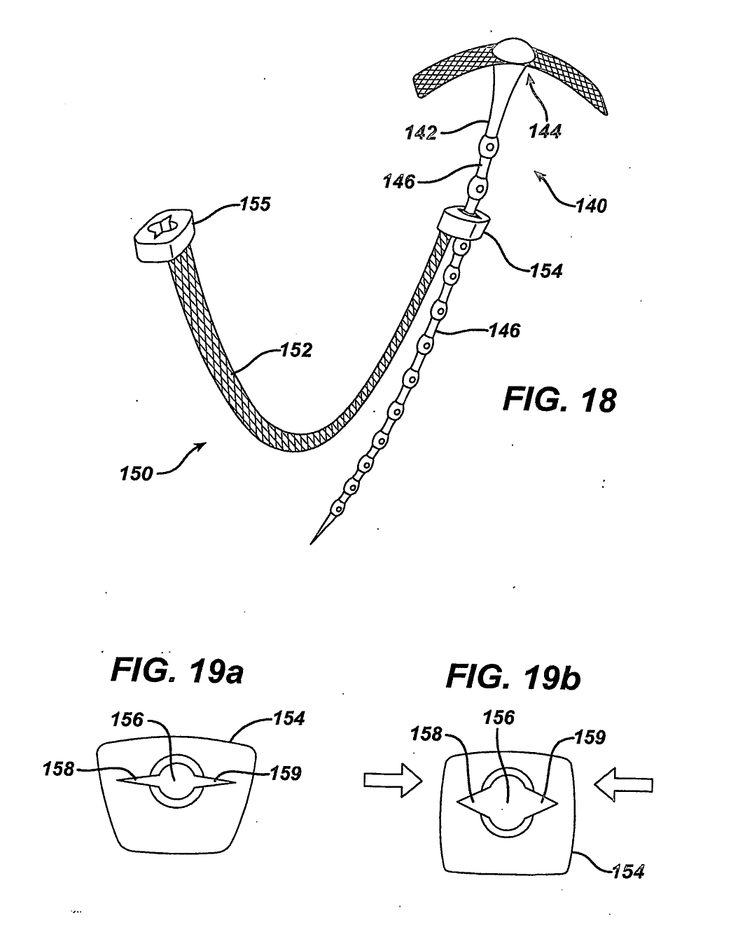

FIG. 18 shows a perspective view of a further embodiment of the implant system, in which view the support structure and a suspending device with deployed tissue anchor are shown,

FIG. 19 shows, in part (a), a plan view of a fastening device according to FIG. 18 in a state in which it is locked on the suspending device, and, in part (b), an illustration of how the fastening device can be unlocked by lateral pressure,

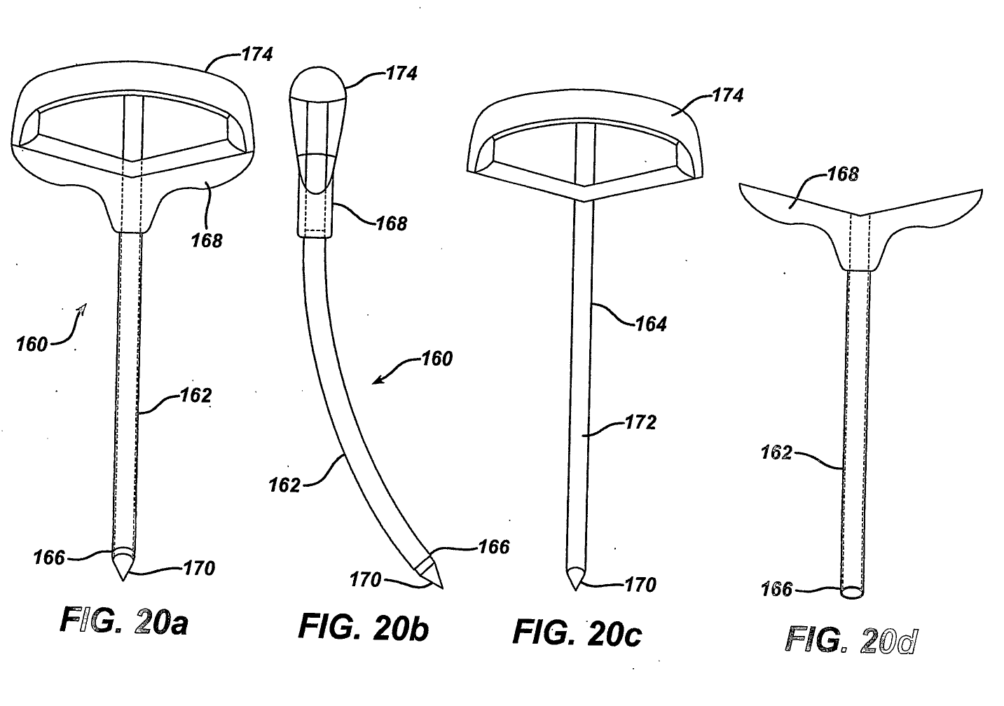

FIG. 20 shows views of an embodiment of a trocar instrument with a sleeve and a trocar for introducing a tissue anchor, specifically, in part (a), a front view of the trocar in the inserted state, in part (b) a side view of the trocar in the inserted state, in part (c) a front view of the separate trocar, and in part (d) a front view of the separate sleeve,

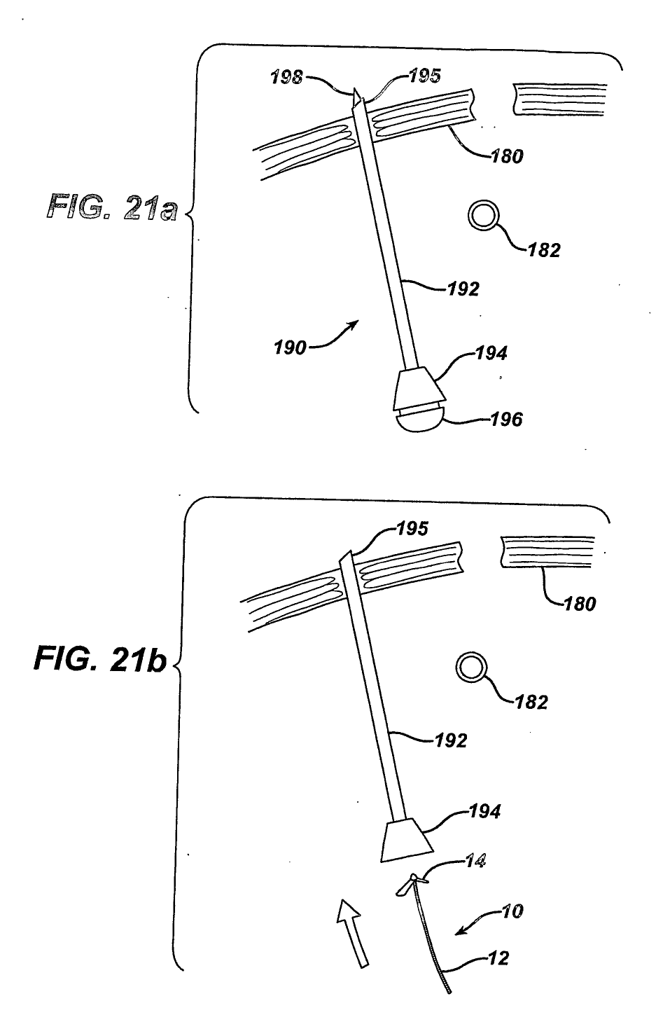

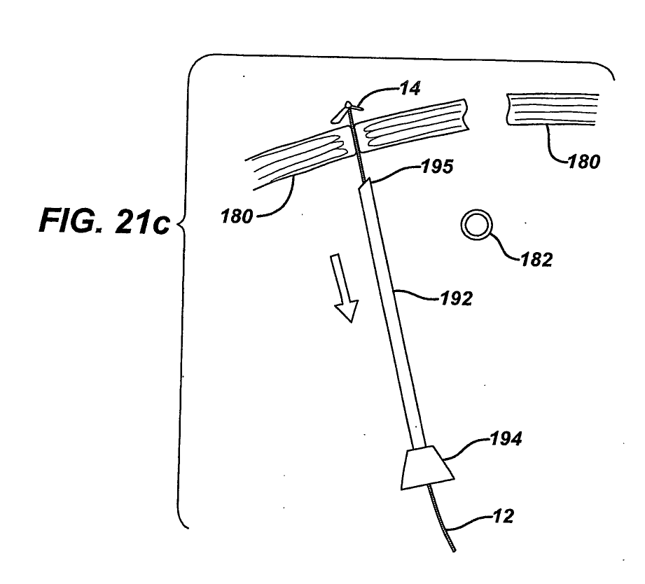

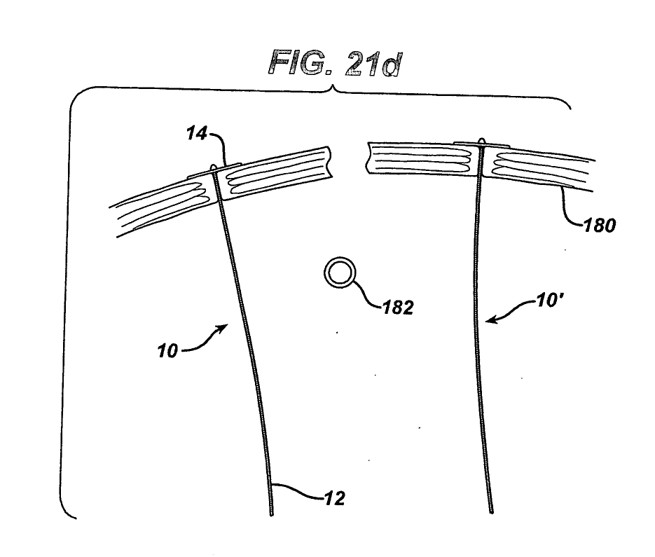

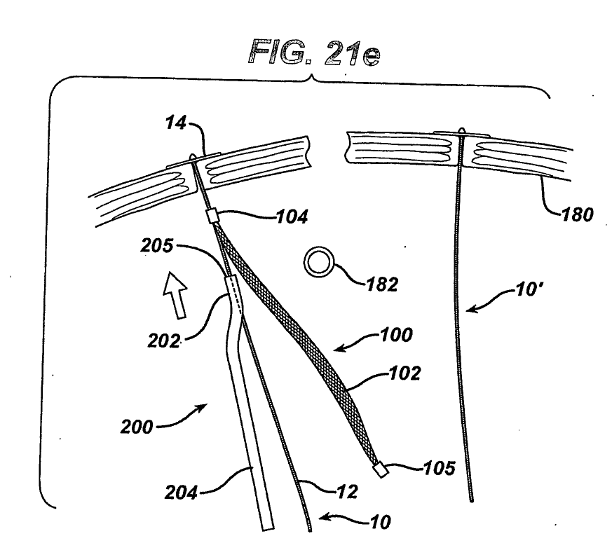

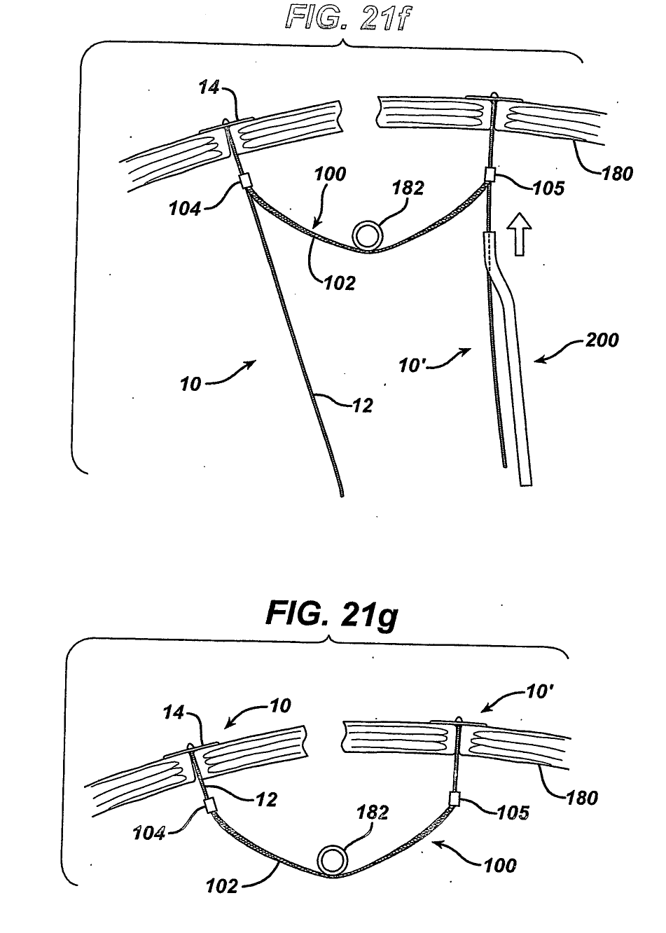

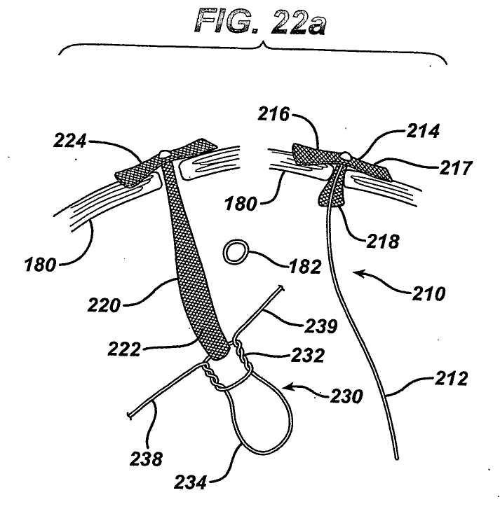

FIG. 21 shows, in parts (a) to (g), diagrammatic views of successive steps in a surgical procedure in which an embodiment of the implant system is fastened on the pubocervical fascia in order to support the urethra, and

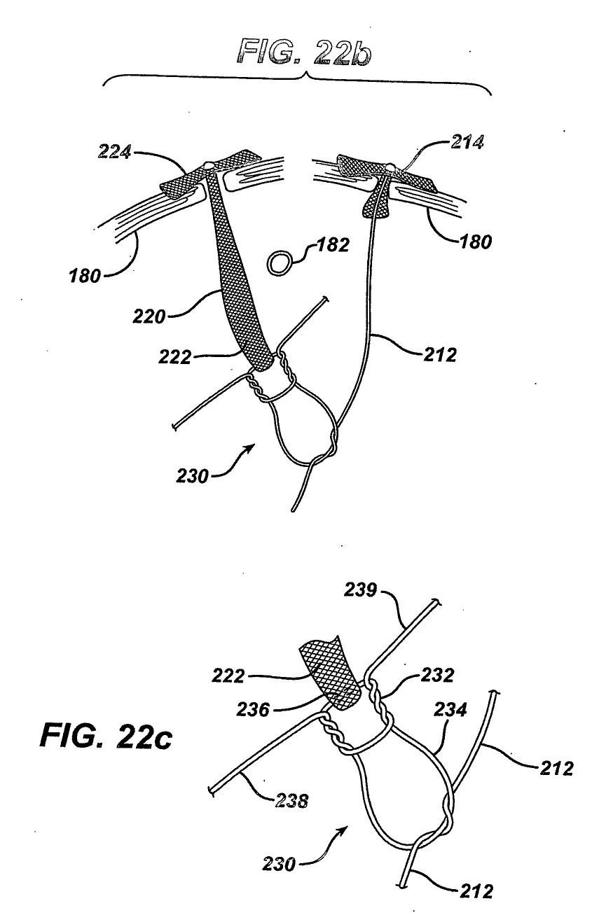

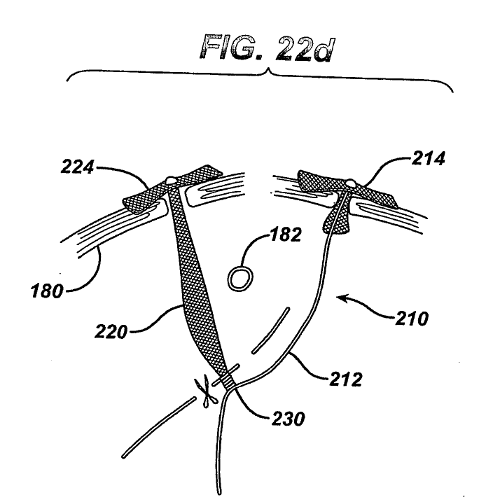

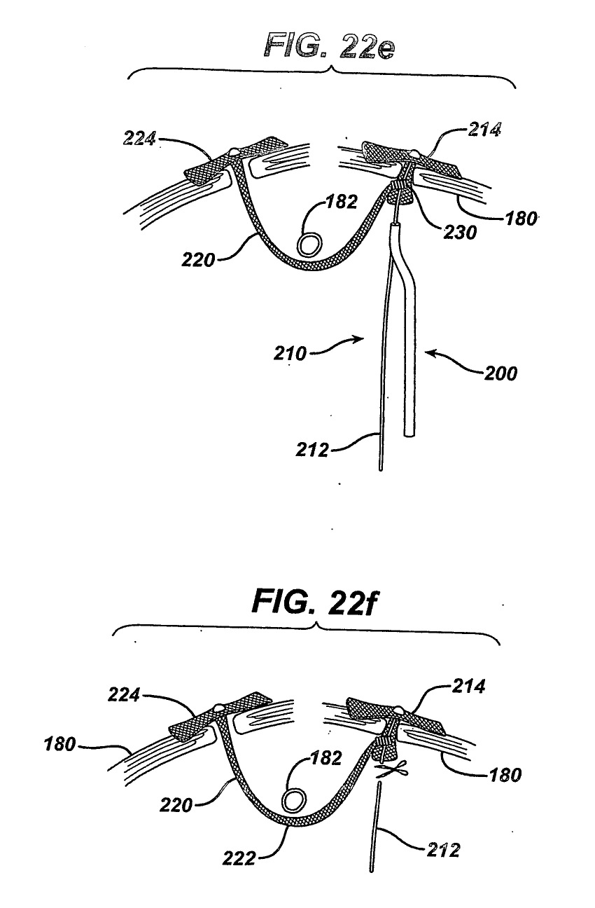

FIG. 22 shows, in parts (a) to (f), diagrammatic views of successive steps in a surgical procedure in which a differently designed embodiment of the implant system is fastened on the pubocervical fascia in order to support the urethra.

To illustrate the invention, embodiments of the individual components of the surgical implant system will first be discussed and it will be explained how the parts are connected to one another. The surgical technique will then be described on the basis of two illustrative embodiments. In the text below, the terns “above”, “below” and “the side of” refer illustratively to the implanted state when the patient is in an upright position.

FIG. 1 shows a perspective view of a suspending device 10 which is a component of the surgical implant system. The suspending device 10 has a threadlike structure in the form of a monofilament 12 with a lower end and an upper end. A tissue anchor 14 is fastened at the upper end of the monofilament 12, as is illustrated in an exploded view in FIG. 2. The tissue anchor 14 has two wings 16 and 17 which in this illustrative embodiment are made integrally from one piece of implant tape, which for example is made of knit polypropylene monofilaments. The wings 16 and 17 lie on a collar 18, and an extension part 20 extends through a central opening at the connection point of the wings 16 and 17. The arrangement is secured by a cap 22 which is locked onto a projection at the end of the extension part 20.

Arranged along the monofilament 12, there are depressions 24 which, in this illustrative embodiment, are equidistant and ring-shaped, and they can also continue farther down than is shown in FIG. 2. They are used to receive a locking projection of a suspending device, as will be explained below.

In the illustrative embodiment, the monofilament 12 is made of polypropylene, has a diameter of 1 mm and is made as a molded part in one piece with the collar 18 (diameter 3 mm) and with the extension part 20. Other materials, sizes and production techniques are also possible, however.

FIG. 3 shows a further embodiment of a suspending device which is here designated by 30. The suspending device 30 has a monofilament 32 and a tissue anchor 34, in which, instead of the wings 16 and 17, a circular disc 36 of flexible tape material is used.

FIG. 4 shows a variant of the embodiment in FIG. 3 in which a three-winged disc 38 of flexible tape material serves as the tissue anchor.

FIG. 5 shows a further embodiment of a suspending device, which is here designated by 40. The suspending device 40 once again has a monofilament 42 and a tissue anchor 44. The tissue anchor 44 is here produced as a molded part with radially extending segments 46. The segments 46 are flexible and can be folded inwards so that they extend substantially parallel to the longitudinal axis of the suspending device 40, as is shown in FIG. 5, part (a). Part (b) shows the deployed state. In this illustrative embodiment, the segments 46 move automatically from the folded-in state to the deployed state.

A further embodiment of the suspending device, here designated by 50, is shown in FIG. 6. Part (a) is an exploded view of the upper area which illustrates how a wing part 54 is held with the aid of a cap 52. The wing part 54 is produced as a molded part and has openings 56.

FIG. 7 illustrates a further embodiment of a suspending device, which is here designated by 60. The suspending device 60 has a monoflalment 62 with ring-shaped depressions 64 which can also continue further down than is shown in FIG. 7. A wing part 66 is molded on via a film hinge 68 and can be pivoted in the direction of the arrows from the transverse position according to part (a) to a position in which it is folded upwards according to part (b), from which it automatically returns to the position according to part (a) when no external forces act on it. FIG. 7 part (c) shows a slightly modified embodiment in which the wing part designated here by 66′ tapers off in thickness towards its two free end, so that the end areas are more flexible than the central area.

FIG. 8 shows an embodiment of a suspending device, here designated by 70, which has a structure similar to the embodiment according to FIG. 1. The suspending device 70 has a monofilament 72 with depressions 73 (see FIG. 10) and, at the upper end, a tissue anchor 74 with two wings 76 and 77 made from one piece of tape. In addition, the tissue anchor 74 has a sleeve-shaped structure 78 of textile material (preferably the material from which the wings 76 and 77 are made) which encloses the upper area of the monofilament 72. The sleeve-shaped structure 78 improves the fit and the growth of the tissue anchor 74 in the patient's body tissue. Since the wings 76 and 77 and the sleeve-shaped structure 78 are porous, tissue can grow through them in the course of the healing process. An atraumatically shaped cap 79 serves as upper closure piece.

In an alternative configuration, the sleeve-shaped structure 78 is replaced by a single wing made of textile material which extends a distance downwards but does not completely enclose the upper area of the monofilament 72.

FIG. 8 also shows a futher component of the surgical implant system, namely a support structure for the urethra, of which support structure a fastening device 80 and part of a tape 82 are shown in FIG. 8. In this illustrative embodiment, the tape 82 is formed as a knit of polypropylene monofilaments.

FIG. 9 shows the fastening device 80 in longitudinal section. The fastening device 80 has a sleeve-like element 84 (external diameter 2.6 mm in this illustrative embodiment) in whose wall a spring tongue 86 is formed which extends slightly downwards and at its lower end has an inwardly directed locking projection 87, see also FIG. 10. The sleeve-shaped element 84 is open at the top and bottom, the upper edge zone 88 being bevelled in a funnel shape. The tape 82 is fastened on the sleeve-shaped element 84 with the aid of a cuff 89, so that the whole external diameter in this illustrative embodiment is about 4.2 mm.

FIG. 10 shows how the sleeve-shaped element 84 of the fastening device 80 is arranged on the monofilament 72. For the sake of clarity, the tape 82 and the cuff 89 have been omitted in FIG. 10. The fastening device 80 can be pushed onto the monofilament 72 over the lower end of the monofilament 72, the funnel-shaped edge zone 88 making engagement easier. The fastening device 80 can be moved along the monofilament 72. When the locking projection 87 comes to lie in a depression 73, the tape 82 is firmly fastened on the suspending device 70. However, under the effect of forces which exceed the forces occurring in the patient's body after completion of the operation, the engagement between the locking projection 87 and the respective depression 73 can be overcome, so that the fastening device 80 can be moved along the monofilament 72 in order to set an optimum position of the tape 82 during the operation.

FIG. 11 shows a variant of the fastening device in which a sleeve-shaped element 90 has a circumferential groove 92. In the variant according to FIG. 12, a sleeve-shaped element 94 has a conically shaped lower area 96. The groove 92 and area 96 improve the fastening of the tape with the aid of a cuff.

FIG. 13 shows embodiments of the support structure in overall view Parts (a), (b) and (c) show support structures 100, 100′ and 100″ with tapes 102, 102′ and 102″ of different length, in each case with two fastening devices 104 and 105 arranged at both ends of the tape. The fastening devices 104 and 105 are of the structural type discussed with reference to FIGS. 8 to 10. Given considerable anatomical differences, it can be advantageous for tapes 102, 102′ and 102″ of different length to be made available so that the implant system can be adapted without problem to the anatomical conditions presented by the particular patient. The width of a tape is preferably as great as that of the tapes used in the TVT technique mentioned at the outset, and is of the order of 10 mm. The optimum length is determined by the surgical technique described below and is several centimeters.

FIG. 14 shows a further embodiment of a fastening device, which is here designated by 110. The fastening device 110 has a ring-shaped element 111 into whose internal area two locking projections 112 and 113 protrude.

The embodiment according to FIG. 15 is of similar construction, in which figure a fastening device 120 with a ring-shaped element 121 is shown which, in this illustrative embodiment, has an external diameter of 4 mm and a thickness of 1 mm. Once again, two locking projections 122 and 123 protrude into the internal area. The ring structure has two notches 124 and 125 lying opposite one another. Offset by 90° to these, two grip recesses 126 and 127 lie opposite one another.

FIG. 16 shows how the fastening device 120 is fastened on one end of a tape 130. If the fastening device 120 is pushed onto a suspending device with a monofilament 132 with depressions 134, the two locking projections 122 and 123 lie in a depression 134, as is illustrated in FIG. 17 part (a). FIG. 17 part (b) shows how, by exerting lateral pressure on the two grip recesses 126 and 127, the ring structure bends, on account of the notches 124 and 125, so that the locking projections 122 and 123 move out from the respective depression 134 and the fastening device 120 is disengaged from the monofilament 132.

FIG. 18 shows a perspective view of a further embodiment of a suspending device, here designated by 140, with a support structure fastened on it. The suspending device 140 once again has a monofilament 142 on whose upper end a tissue anchor 144 is arranged and which is provided in its longitudinal direction with depressions 146, which, however, are differently formed than the depressions in the embodiments considered above.

The support structure designated by 150 has a tape 152 at whose ends fastening devices 154 and 155 of identical structure are arranged. FIG. 19 part (a) shows the fastening device 154 in a plan view. It has a central opening 156 through which the monofilament 142 is passed and from which two slits 158 and 159 issue. If the fastening device 154 is compressed in the direction of the two arrows indicated in FIG. 19 part (b), the fastening device 154, made of flexible material, deforms to such an extent that the opening 156 assumes a circle-like shape, with the result that the fastening device 154 can be moved out of a respective depression 146 on the monofilament 142. If these pressure forces do not occur, the fastening device 154 is by contrast locked securely in a depression 146.

FIG. 20 shows different views of a trocar instrument 160 which, as a further component of the surgical implant system, can be used, during an operation to insert the support structure, in order to carefully establish an opening in a internal anatomical tissue structure in order to pass a tissue anchor of said type through this opening and suspend the suspending device thereon.

The trocar instrument 160 has a sleeve 162 and a trocar 164. In parts (a) and (b), the trocar 164 is inserted fully into the sleeve 162, and part (b) shows that the trocar instrument 160 is curved in this illustrative embodiment. In parts (c) and (d), the trocar 164 and the sleeve 162 are shown separately. The sleeve 162 has a distal end 166 and, in its proximal end area an ergonomically shaped handle 168. When the trocar 164 is inserted completely into the sleeve 162, its tip 170 protrudes from the distal end 166 of the sleeve 162. The trocar 164 has a shaft 172 which issues from a likewise ergonomically shaped handle 174 When the trocar 164 is inserted into'the sleeve 162, the handles 168 and 174 also bear on one another, resulting in what is overall a favourably shaped handle.

FIG. 21 parts (a) to (g) are diagrammatic illustrations of the successive steps in an operation for implanting a support structure for the urethra. In this example, the implant system uses a support structure 100 with two fastening devices 104 and 105 (see FIGS. 9, 10 and 13) fastened on two suspending devices 10, 10′ of the structure shown in FIGS. 1 and 2. The arrangement is fastened on the pubocervical fascia, which is designated by 180 in FIG. 21 The urethra 182 is also indicated in cross section. The other anatomical structures have been omitted for the sake of clarity. Part (a) shows how, with the aid of a trocar instrument 190 introduced through an incision in the upper vaginal wall, an opening is established in the pubocervical fascia 180. In this example, the trocar instrument 190 is of a slightly different design than the trocar instrument 160 and has a sleeve 192 with a handle 194, the distal end 195 of the sleeve being bevelled. A trocar 196 with a tip 198 is inserted into the sleeve 192. In the state shown in part (a), the tip 198 has bored through the pubocervical fascia.

The trocar is thereafter withdrawn, and a suspending device 10 can be introduced into the sleeve 192 with the tissue anchor 14 leading, as is indicated by the arrow in part (b). The two wings 16 and 17 of the tissue anchor 14 fold together.

When the tissue anchor 14 emerges at the distal end 195 of the sleeve 192, the wings 16 and 17 deploy and thereby anchor the suspending device 10 on the pubocervical fascia 180. The sleeve 192 can now be withdraw in the direction of the arrow in FIG. 21 part (c). In this way, the suspending device 10 is fastened to the left side above the urethra 182.

In the same way, a further fastening device, here designated by 10′, can be suspended with the aid of a tissue anchor to the right side above the urethra 182, see part (d).

The fastening device 104 of the support structure 100 is now pushed onto the monofilament 12 of the suspending device 10 via the lower end of the monofilament 12 of the suspending device 10. A sliding instrument 200 is used for further advancing it, see part (e), which sliding instrument is made of a tube 202 with a proximal area 204 and a distal end 205. The tube 202 is bent at a right angle and, opposite its opening at the distal end 205, it has a further opening at the bend position 206, so that the monofilament 12 can be guided through these two openings. The proximal area 204 serves as a handle of tee sliding instrument 200. The distal end 205 engages on the fastening device 104 and permits an advance movement in the direction towards the tissue anchor 14. In doing so, the locking projection of the fastening device 104 passes in succession through the depressions in the monofilament 12, as has been explained above, and fastens the support structure 100 when the fastening device 104 has reached its final position.

As is shown in part (f), the fastening device 105 is then engaged in an analogous manner into the monofilament of the suspending device 10′ and pushed upwards by the sliding instrument 200 until the tape 102 of the support structure 100 has reached an optimum position. In the view according to part (f), the tape 100 lies directly on the urethra 182. In practice, however it is more advantageous if the urethra 132 is only loosely supported, so that the tape 100 only touches the urethra 182 in stress situations. A stress situation can be simulated during the operation by asking the patient to cough.

If the fastening devices 104 and 105 have been pushed too high, it is possible to pull them back down again slightly, using an instrument not shown in the figures, in order thereby to correct the position of the support structure 100 under the urethra 182.

Finally, the protruding ends of the monofilament 12 of the suspending device 10 and of the monofilament of the suspending device 10″ are cut off. FIG. 21 part (g) shows the final state of the implant.

FIG. 22 illustrates the use of an alternative embodiment of the implant system on the basis of successive surgical steps. In this embodiment of the implant system, only one suspending device is used, while the support structure has a lengthened tape used for anchoring on the other side of the urethra. In this example too, the implant is suspended on the pubocervical fascia 180; the urethra is once again indicated by 182.

FIG. 22 part (a) shows how a suspending device 210 with a monofilament 212 is anchored with the aid of a tissue anchor 214 with two wings 216 and 217 and a downwardly extending tape section 218 to the right above the urethra 182 in the pubocervical fascia 180.

Fastened to the left side above the urethra 182, specifically by means of a tissue anchor 224, there is a tape 220 whose lower area is provided as a support structure 222 for the urethra 182. In the same way as was described with reference to FIG. 21, the suspending device 210 and the tape 220 can be anchored in the pubocervical fascia 180 with the aid of a trocar instrument.

A double loop 230 is used as the fastening device for fastening the support structure 222 on the suspending device 210. The double loop 230 comprises a first loop 232 and a second loop 234. The first loop 232 is pulled through the lower edge area 236 of the support structure 222 (i.e. of the tape 220). Issuing from here there are two free end portions 238 and 239, with which the double loop 230 as a whole can be pulled tight.

As is illustrated in part (b), the monofilament 212 of the suspending device 210 is passed twice through the second loop 234. Part (c) is an enlarged detail from part (b) and shows the course of the thread in the area of the double loop 230 and of the monofilament 212.

By pulling the two end portions 238 and 239, the double loop 230 as a whole is pulled closed, so that the monofilament 212 is fastened on the edge area 236 of the support structure 222. The two end portions 238 and 239 can optionally be knotted in order to obtain a still more secure fastening. The two end portions 238 and 239 are then cut, as is indicated in part (d).

The nature of the double loop 230 makes it possible for the edge area 236 of the support structure 222 to be moved upwards even when it is still in the state according to part (d). Use can once again be made of the sliding instrument 200 discussed with reference to FIG. 21, or of a similar slide instrument, see part (e). If so required, the edge area 236 of the support structure 222 can also be pulled back down again slightly.

When the optimum position in relation to the urethra 182 is reached, the excess end of the monofilament 212 is cut off, see FIG. 22 part (f).

In this illustrative embodiment, the suspending device 210 has one monofilament 212. In the illustrative embodiments considered above, one monofilament is preferred because one monofilament can be more easily provided with depressions. In the illustrative embodiment according to FIG. 22, however, another thread structure could also be used on the suspending device 210, for example a braided thread. The friction between the closed double loop 230 and the threadlike structure of the suspending device 210 determines how firmly the support structure 222 is fastened on the suspending device 210 and how easily it can be moved along the threadlike structure. This fiction can be influenced by the nature of the thread material, but also by the number of loops used. There are therefore numerous possible variants of the illustrative embodiment described.

Before cutting off the excess portion of the suspending device 210, additional securing can be obtained if a sliding knot on the threadlike structure (that is to say on the monofilament 212 in this illustrative embodiment) is moved up until it bears on the closed double loop 230. The sliding instrument 200 or a similar instrument can once again be used for this purpose.

The many described embodiments of the implant system show that the basic concept of fastening a support structure for the urethra on a suspending device, such that it can still be moved along the suspending device in order to adjust the position of the support structure, can be realized in a wide variety of ways. Many further variants are therefore conceivable. For example, a fastening device does not have to have a locking projection which bears in depressions on the suspending device, and instead embodiments are also possible in which the fastening device has a clamping mechanism, for example, which can be moved steplessly along a smooth or rough surface of the suspending device with suitable application of pressure. Moreover, tissue stutures other than the pubocervical fascia can also be considered for fastening the support structure.

1. A surgical implant system for treating female urinary incontinence, the system comprising:
at least one suspending device having an upper end and a lower end, the upper end capable of being fastened on an internal anatomical tissue structure to the side of and above the urethra, and
a urethral support structure for implantation transversely relative to the urethra, the support structure comprising a fastening device removeably attached to the suspending device for movement along the upper end of the suspending device to adjust the position of the support structure.

2. The system according to claim 1, wherein the suspending device comprises a threadlike structure.

3. The system according to claim 2, wherein the threadlike structure is a monofilament.

4. The system according to claim 3, wherein the monofilamnent has at least one depression embedded therein, the depression being engageable with at least one locking projection on the fasting device.

5. The system according to claim 4, wherein the fastening device comprises at least one spring tongue, and the at least one locking projection is arranged thereon.

6. The system according to claim 5, wherein the fastening device comprises two locking projections opposite each other and moveable away from each other by lateral pressure exerted on the fastening device.

7. The system according to claim 1, wherein the at least one spending device further comprises a tissue anchor.

8. The system according to claim 7, wherein the tissue anchor further comprises at least two wings.

9. The system according to claim 8, wherein the wings are movable from a configuration extending substantially parallel to the longitudinal axis of the at least one suspending device to a configuration extending transversely with respect to the longitudinal axis of the at least one suspending device.

10. The system according to claim 8, wherein the wings are made of tape-like textile material.

11. The system according to claim 8, wherein the tissue anchor has a sleeve-shaped structure of textile material under the wings.

12. The system according to claim 1, further comprising a trocar instrument for introducing the at least one suspending device.

13. The system according to claim 12, wherein the trocar instrument has a sleeve with a proximal end and a beveled distal end.

14. The system according to claim 13, wherein the trocar instrument is curved.
